(19) United States
(12) Patent Application Publication
(10) Pub. No.: US 2015/0131240 A1
(43) Pub. Date: May 14, 2015

(54) Method for Producing an Electronic Subassembly

(71) Applicant: Jan Kostelnik, Kichberg (DE)

(72) Inventor: Jan Kostelnik, Kichberg (DE)

(73) Assignee: Würth Elektronik Gmbh &Co. KG, Niedernhall (DE)

(21) Appl. No.: 14/402,095

(22) PCT Filed: May 22, 2012
(86) PCT No.: PCT/EP2012/059476

§ 371 Date: Dec. 19, 2014

(51) Int. Cl. B32B 37/14; A61N 1/375; H04R 25/00; A61M 5/142; H05K 1/18; H05K 1/09; B32B 38/10; A61N 1/362

(52) U.S. Cl. CPC B32B 37/144; B32B 38/10; A61N 1/375; A61N 1/362; A61M 5/14276; H05K 1/189; H05K 1/09; H04R 25/606; B32B 2307/202; B32B 2305/80; H05K 2203/1131
USPC 361/751; 156/47; 216/20

(57) ABSTRACT

The invention concerns a method for production of electronic assembly (1) with

1.1 Supply of an electrically-conducting film (3), especially a support film (3a),

1.2 Supply of at least one electrical component (5) with at least one electrical contact site (5c),

1.3 Application of an adhesive (20) between the electrical component and a surface (30) of the electrically-conducting film,

1.4 Arrangement of the at least one component (5) with the at least one electrical contact site (5c) on the surface (30) of the electrically-conducting film (3) and fastening of the at least one component by formation of an adhesive joint between the electrical component and the surface,

1.5 Supply of the support (9), especially from a flexible material,

1.6 Lamination of the film (3) with support (9) so that the at least one electrical component (5) is arranged between film (3) and support (9) and formation of a mechanical and electrical connection (23) between the electrical contact site of the at least one electrical component (5) and the electrically-conducting film (3) by low-temperature sintering of nanoparticles, especially from gold, silver, nickel or copper or from an alloy of these metals, in which lamination of the film occurs simultaneously with low-temperature sintering,

1.7 Structuring of the electrically-conducting film (3) to conductor tracks (11) and/or cooling surfaces (13).

The invention concerns a method for production of an electronic assembly and an electronic assembly.

Such a method is known from DE 10 2008 009 220 A1 and from DE 10 2010 014 579 A1.

The task of the present invention is to provide an improved method and an improved electronic assembly.

This task is solved in terms of the method by

1.1 Providing an electrically-conducting film, especially a support film,

1.2 Providing at least one electrical component with at least one electrical contact site,

1.3 Application of an adhesive between the electrical component and the surface of the electrically-conducting film,

1.4 Arrangement of at least one component with at least one electrical contact site on the surface of the electrically-conducting film and fastening of the at least one component by formation of an adhesive joint between the electrical component and the surface,

1.5 Providing a support, especially from a flexible material,

1.6 Lamination of the film with the support so that the at least one electrical component is arranged between the film and support and formation of mechanical and electrical connection between the electrical contact site of the at least one electrical component and the electrically-conducting film by low-temperature sintering of nanoparticles, especially from gold, silver, nickel or copper or from an alloy of these metals, lamination of the film occurring simultaneously with low-temperature sintering,

1.7 Structuring of the electrically-conducting film to conductor tracks and/or cooling surfaces.

Variant [sic; variants (Ausführungformen)] of the invention are particularly advantageous, since lamination and low-temperature sintering can occur in the same process step essentially simultaneously. This is made possible, on the one hand, by using nanoparticles, which permit low-temperature sintering in a temperature range that is also suitable for lamination of the film and, on the other hand, by previous fastening of the at least one electrical component on the surface of the electrically-conducting film. By this fastening it is ensured that the electrical component is situated in the correct location when the support for subsequent lamination is applied to the film and the component situated in the space and that the component cannot slide.

“Electrical component” is understood to mean here especially housed and unhoused electronic components, like integrated semiconductor chips, for example, so-called bare chips (or bare dies), as well as discrete electrical or electronic components, like capacitors.

Such an electrical component ordinarily has a side on which one or more electrical contact sites are provided for electrical contacting of the component. This side of the component is also referred to as “active side” of the component. In particular, if the component is a bare die, the electrical contact sites are formed as so-called contact pads.

The support can be preimpregnated fibers (i.e., a so-called prepreg), which are understood to mean here an uncured thermosetting plastic matrix. For example, the support can be a liquid crystalline polymer (LCP), which is particularly advantageous for production of biocompatible implants, or another support material.

According to one embodiment of the invention an electrically conducting film whose surface is coated with nanoparticles is used as starting material. The at least one electrical component is then fixed on the surface of the electrically-conducting film by an adhesive, which is applied outside the electrical contact sites of the component. The adhesive is preferably a nonconducting adhesive, i.e., a so-called a nonconducting adhesive (NCA) or nonconducting paste (NCP). If the electrical component is a bare die, which is surrounded outside the electrical contact sites by a passivation layer, the adhesive joint is produced by means of this adhesive between the passivation layer and the surface of the electrically-conducting film.

According to one embodiment of the invention, an electrically-conducting film whose surface need not be coated with nanoparticles is used as starting material. In this case an adhesive containing the nanoparticles is used. The adhesive is applied to the at least one electrical contact site of the electrical component in order to fix the electrical component on the surface of the electrically-conducting film. The support is then applied in order to laminate the film with the support and at the same time conduct low-temperature sintering. In this case the nanoparticles contained in the adhesive joint are sintered. This embodiment of the method according to the invention is particularly advantageous, since the nanoparticles that are required for subsequent low-temperature sintering are also applied simultaneously by application of the adhesive.

According to one embodiment of the invention low-temperature sintering is conducted as “pressureless” low-temperature sintering. “Pressureless” is understood here to mean that low-temperature sintering is not conducted at the usually employed high pressures of, say, 200 bar, but at a much lower pressure, as is also used for lamination, i.e., at a pressure of, say, 15-20 bar.

Appropriate adhesives or pastes that contain the nanoparticles for such pressureless low-temperature sintering are known per se from the prior art, for example, from DE 10 2008 039 828 A1 and are commercially available from Heraeus under the trade names mAgic Adhesive and mAgic Paste.

A particularly efficient and material-saving production method is made possible on this account, since the amount of employed nanoparticles can be minimized. The joint so produced is also particularly mechanically stable and has good electrical contact properties owing to the covalent bonds of the nanoparticles that lead to limited contact resistance.

According to one embodiment of the invention the size of the nanoparticles is chosen so that they are relatively chemically inert at room temperature so that no spontaneous sintering occurs at room temperature. On the other hand, the size of the nanoparticles can be chosen sufficiently small so that the reactivity of the nanoparticles in comparison with macroscopic particles is already increased in the temperature range required for lamination so that formation of covalent chemical bonds occurs between the nanoparticles so that they are sintered to each other.

According to one embodiment of the invention the adhesive can be an LCP. This is particularly advantageous if the support is also formed by an LCP, especially with respect to thermal loadability and long-term stability of the resulting electronic assembly, which is particularly advantageous for application in the medical field, especially for implants.

In another aspect the invention concerns a method for production of an electronic assembly in which before lamination of the film with the support the at least one component arranged on the electrically-conducting film is enclosed with a filler material from a polymer, in which case low-temperature sintering already occurs in this enclosure step. This is particularly advantageous, if the resulting intermediate product must be transported from one production stage to another in a discontinuous process and also for the mechanical stability of the resulting electronic assembly.

In a further aspect the invention concerns an electronic assembly which was produced according to the method of the invention. This can be an implant, like an electronic module for an insulin pump, a pacemaker or an implantable hearing aid.

Provision of the film advantageously occurs by supplying individual foil sections cut to size or by supplying an endless film from a roll.

Provision of the at least one electrical component preferably occurs by supplying it on an endless film or supply in a magazine.

Arrangement of the at least one electronic component preferably occurs in automated fashion with an insertion robot or by hand, especially by means of templates or position markers in or on the film.

Provision of the support can occur by supplying individual support sections cut to size or by supplying an endless support from a roll.

Lamination of the film with the support preferably occurs by exerting a contact pressure on the film in the direction of the support, especially with simultaneous influence of heat.

Structuring of the electrically-conducting film to conductor tracks and optionally cooling surfaces advantageously occurs by direct structuring, for example, with a laser, or by generation of a positive or negative mask and subsequent etching.

The film is advantageously wound on a roll with the support and at least one electrical component after structuring of the film.

It can be prescribed in a modification of the invention that enclosure of at least one electrical component occurs after arrangement of the component on the film with a filler material from the polymer, especially a thermoplastic, thermosetting plastic or elastomer.

Filling of the filler advantageously occurs in predefined form, especially by casting, foaming, extrusion or lamination.

In a modification of the invention it can be prescribed that generation of at least one additional support layer occurs on the conductor tracks generated by structuring, in which case this is preferably insulating and/or dielectric and/or it contains additional conductor tracks.

In particular, application of at least one reinforcement layer, preferably several reinforcement layers on the support or support layer can occur after step 1.6. It can also be prescribed as an alternative that the support is optionally removed after step 1.6.

In a modification of the invention cutting out of the support in a predetermined shape can occur to form the electronic assembly, especially before and/or after lamination with the circuit board in step 1.6.

The electrically-conducting film can then consist of metal, especially copper, electrically-conducting plastic or electrically-conducting ceramic, on whose surface a layer of silver or a silver compound and/or a three-dimensional structure from metallic nanoparticles is optionally present at least in areas and is optionally reinforced by a support film.

It can be prescribed in particular that the electrically-conducting film on the side facing the at least one electrical component be smooth or roughened or have a bump structure.

It can be prescribed that the at least one electrical component is a passive or active component, especially a chip, in which case it is provided preferably outside the contact sites with a protective coating, especially a lacquer, i.e., a so-called passivation layer.

In a modification it can be prescribed that the protective coating be electrically insulated and/or a dielectric. The support can advantageously be a film, especially from metal, plastic or ceramic, a mat from natural materials, for example, plant fibers, a leather section, a textile section or section from another fabric or composite material or a circuit board, ceramic support or glass support.

In a modification of the invention it can be prescribed that the support is given a reinforcement layer and especially one made of metal, plastic or ceramic, from natural materials, like plant fibers, from leather, from textile or another fabric or composite material or a circuit board, ceramic support or a glass support.

The at least one electrical component can have at least one contact side in which at least one electrical contact site is arranged, in which case the at least one electrical contact site is preferably plane with the corresponding contact side.

The contact site advantageously consists of copper or a copper compound, on whose surface a layer of silver or a silver compound and/or a three-dimensional structure from metallic nanoparticles can optionally be present.

In a modification it can be prescribed that a section of the electrically conducting film is formed as a heat-removing contact area (film cooling section) on the at least one electrical component.

In a modification of the invention it can be prescribed that a cooling element or cooling channel is connected to the film cooling section and/or the at least one electronic component.

Additional details, features and advantages of the invention are apparent from the patent claims, whose wording becomes a content of the description by reference, the following description of preferred embodiments of the invention and with reference to the drawings.

In the drawings:

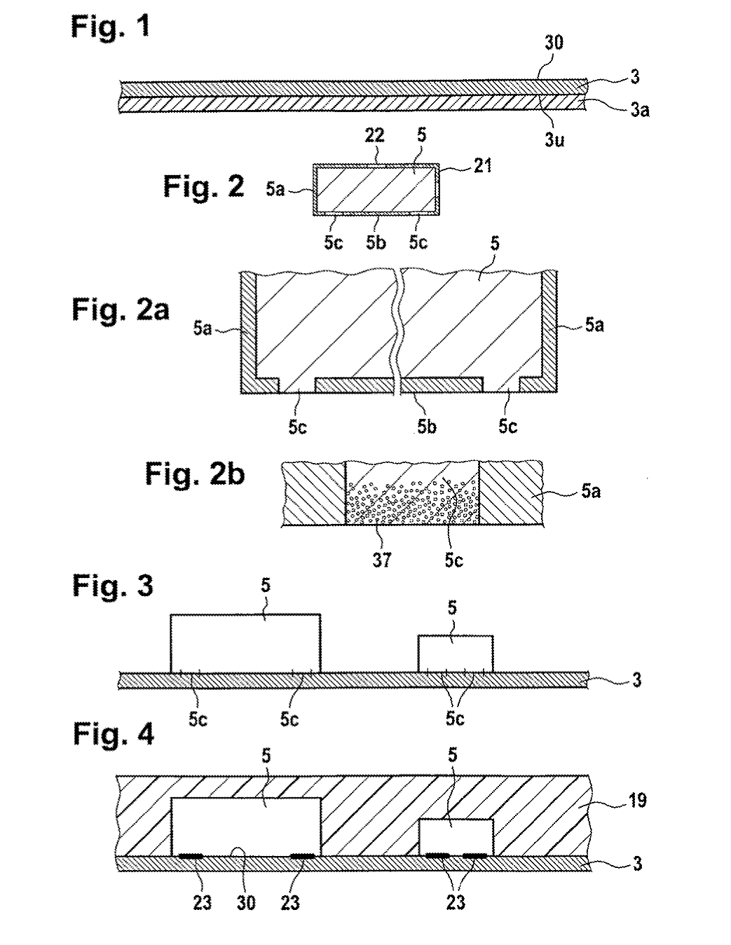

FIG. 1 schematically depicts the side view of an electrically-conductive film as starting material for the method;

FIG. 2 schematically depicts a cross section of electrical components;

FIG. 2a shows an enlarged section of FIG. 2;

FIG. 2b shows an enlarged depiction of a contact site of the component of FIG. 2a;

FIG. 3 shows a side view of the film of FIG. 1 with two components arranged on it;

FIG. 4 shows the arrangement of FIG. 3 in which a filler material is additionally applied;

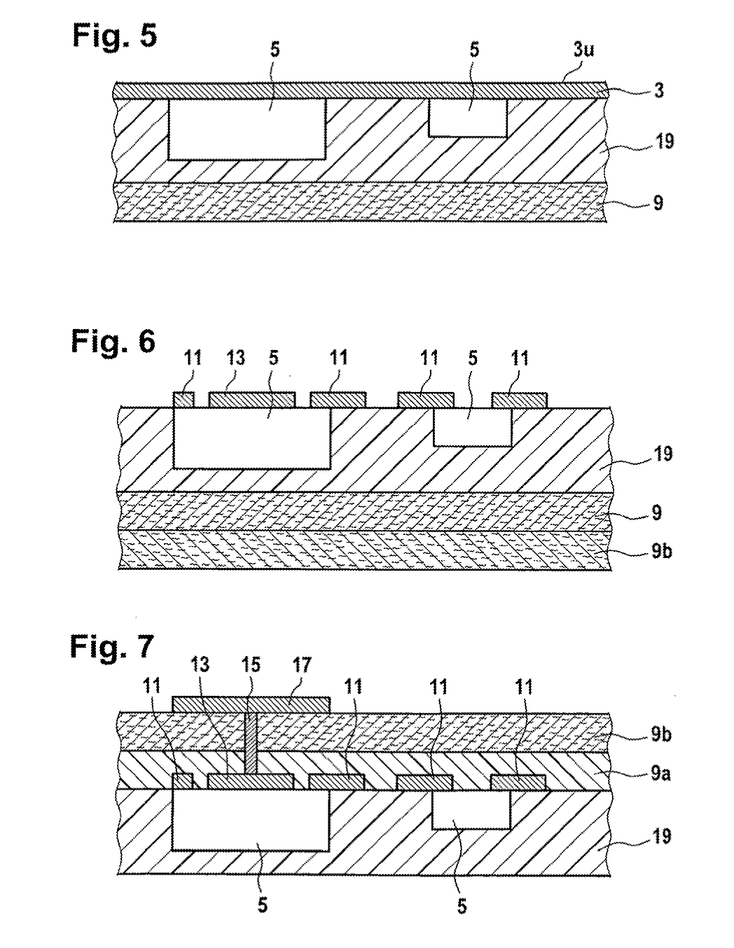

FIG. 5 shows the arrangement of FIG. 4 in which a support is additionally laminated;

FIG. 6 shows the arrangement of FIG. 5 in which conductor tracks and a film cooling section are additionally arranged;

FIG. 7 shows the arrangement of FIG. 6, in which conductor tracks and a film cooling section are additionally arranged but in which the support 9 is removed again and a support 9a is laminated with an additional reinforcement layer 9b on the side of the conductor tracks; and

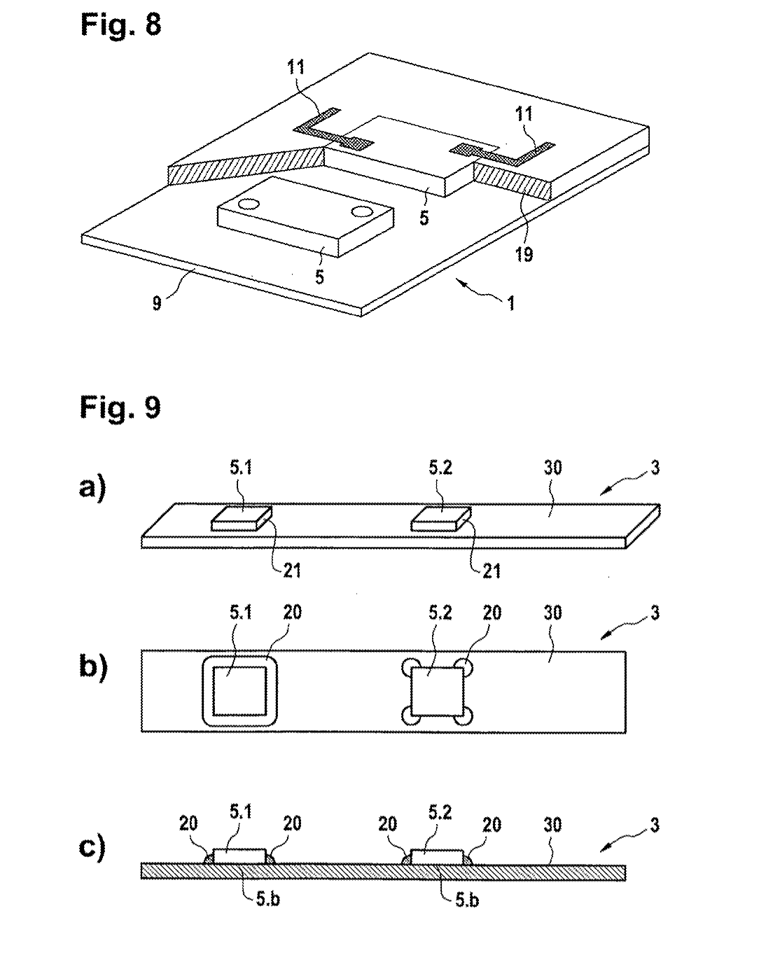

FIG. 8 shows a schematic view of an electronic assembly;

FIG. 9 shows a perspective view of the adhesive joints;

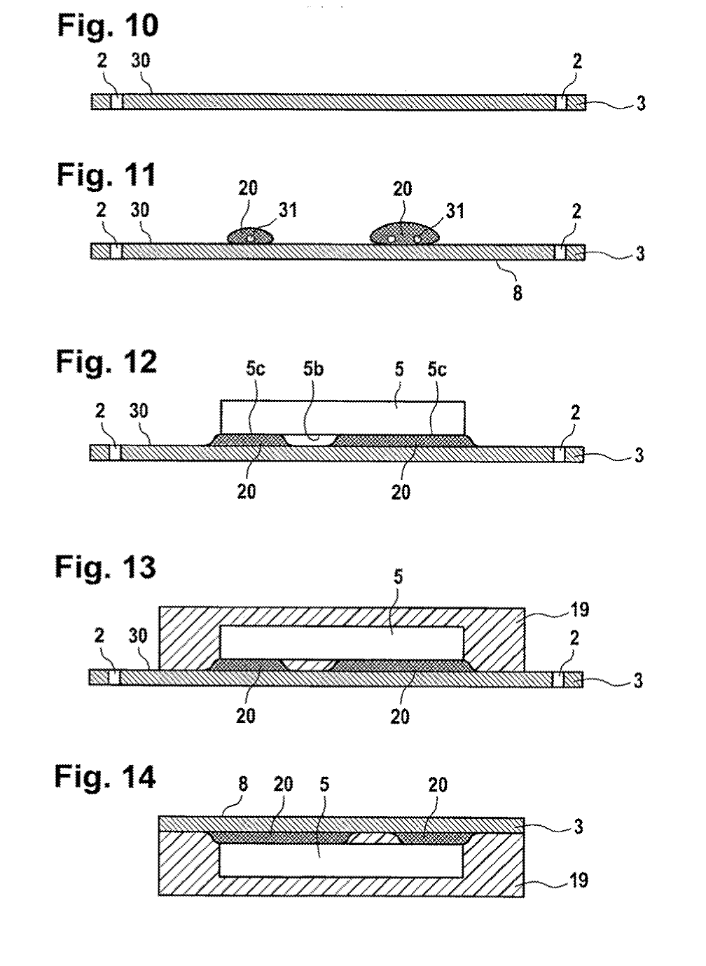

FIG. 10 schematically depicts a side view of a conducting film as starting material for another embodiment of the method according to the invention;

FIG. 11 shows the same section of the film with the applied adhesive points;

FIG. 12 shows the same view with two electronic components glued by means of the adhesive points;

FIG. 13 shows the state of production after application of an enclosure;

FIG. 14 shows the state after cutting out of an electronic assembly;

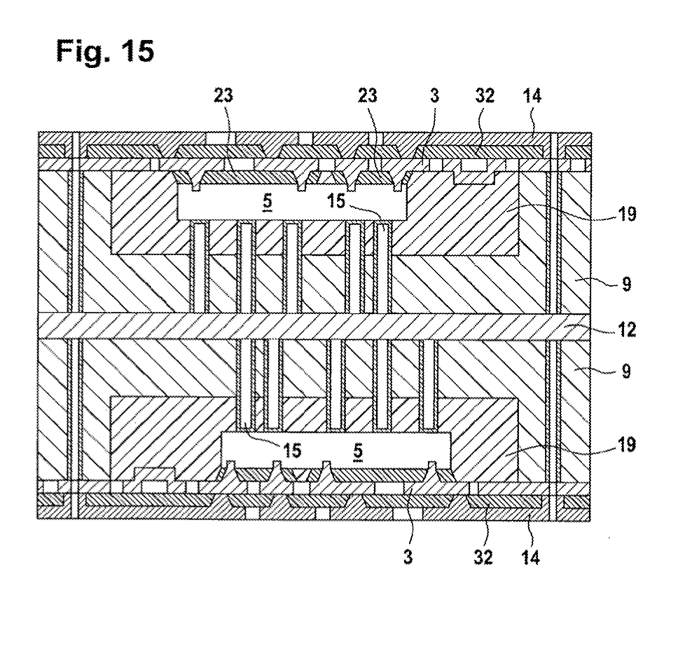

FIG. 15 shows the final state of a multilayer circuit board produced according to the method;

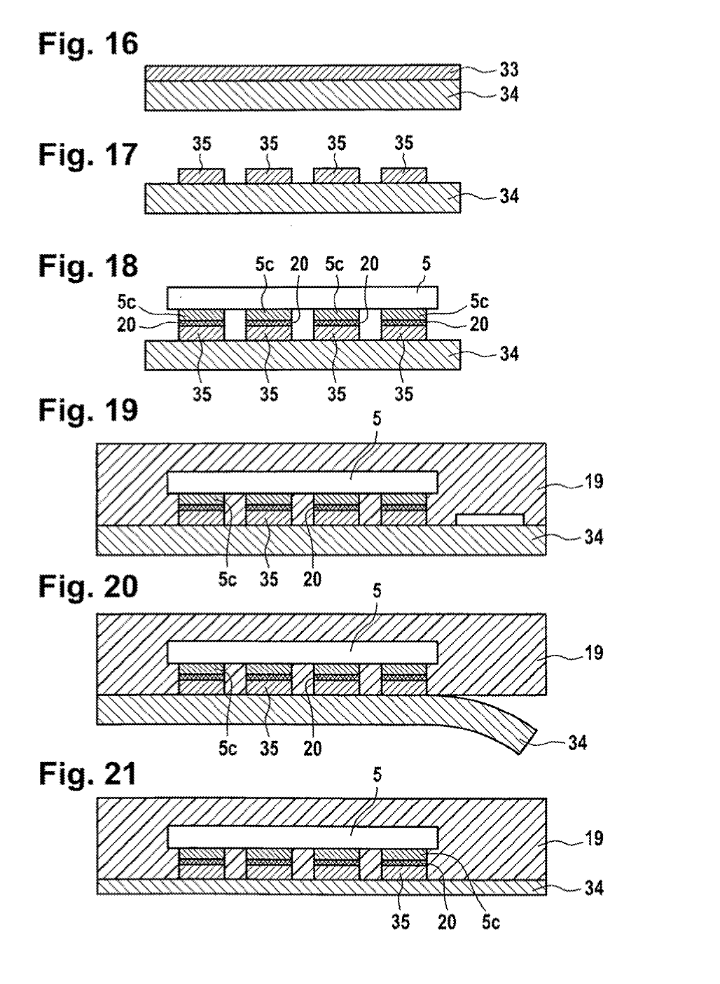

FIG. 16 shows the schematic side view of the starting material according to another embodiment of the method according to the invention;

FIG. 17 shows the state after an additional process step;

FIG. 18 shows the state after an additional process step;

FIG. 19 shows an additional process step;

FIG. 20 shows pulling off of the support film;

FIG. 21 shows a depiction corresponding to FIG. 20 in a modification;

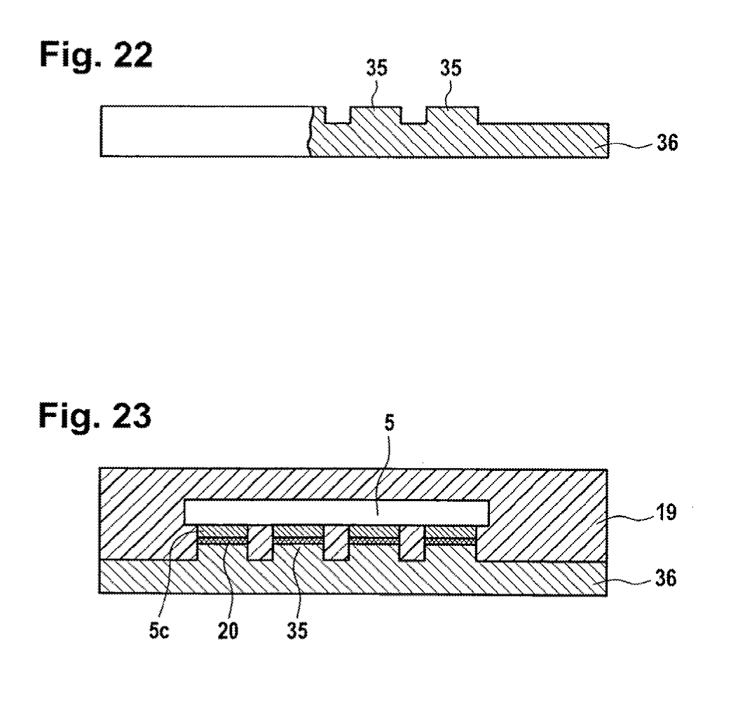

FIG. 22 shows an initial situation in still another embodiment;

FIG. 23 shows the state corresponding to FIG. 21 of the embodiment according to FIG. 22.

Corresponding or identical elements of the different embodiments are subsequently marked with the same reference numbers.

FIG. 1 schematically depicts a section of a side view of an electrically-conducting film 3 of copper unwound from a roll. A film surface 30, which subsequently faces an electrical component 5, is provided with a uniform coating of nanoparticles, for example, saver particles with a particle size of about 20-300 nm or a silver-containing alloy. The film 3 is reinforced on the side opposite side 30 by a support film 3a, which is laminated onto film 3 and consists of a polymer.

FIG. 2 schematically depicts an electrical component, in the present case a chip, in a sectional view. The component 5 is coated on all sides by a protective layer 5a of electrically-conducting lacquer, i.e., a so-called passivation layer. The component 5 has a contact side 5b, which is also referred to as active side with two contact sites 5c for electrical contacting with film 3, which are protected by the applied protective layer 5a. Component 5 can also have additional contact sites, for example, on its edge 21 or on the top 22. The surfaces of contact sites 5c, as is apparent from FIG. 2b, are coated with a layer of silver (a silver-containing alloy), namely with nanoparticles 37. The nanoparticles can partially diffuse into the contact sites 5c, which produces a particularly low electrical contact resistance. The components 5 are prepared from a film unwound from a roll, on which they are arranged at a spacing from each other. For example, the component 5 can be a bare die, in which case the contact sites 5c are then formed as so-called contact pads.

A robot arm grasps one or more components 5 from the film and places them on defined sites on the coated surface 30 of film 3. An adhesive is then applied between component 5 and the surface 30 of electrically-conducting film in order to fix the component by formation of an adhesive joint, in which case joint 7 is an adhesive joint.

The adhesive can be applied before positioning of one of the components 5 on the surface 30 of the film, As an alternative or in addition the adhesive can also be applied robotically, outside the electrical contact sites 5c on the active side, i.e., the contact side 5b of component 5. Another possibility is that the component is initially positioned by the robot arm on the surface 30 of the electrically-conductive film in order to arrange the component there and that subsequently the adhesive to produce the adhesive joint is applied, for example, by applying the adhesive along the side edge of the component (cf. also the embodiments according to FIG. 9).

The adhesive joint produced in this way fixes the component 5 mechanically on film 3 without contacting it electrically. In this embodiment the adhesive 20 (cf. FIG. 9) is preferably applied outside the electrical contact sites 5c so as not to hamper subsequent formation of the mechanical and electrical connection 23 (cf. FIG. 4) by low-temperature sintering, especially since the adhesive 20 in this embodiment contains no metallic nanoparticles.

FIG. 3 shows the arrangement of two components 5 fastened in this way on film 3.

According to one embodiment of the invention a film of thermoplastic polymer is unwound from an additional roll and placed on the arrangement of FIG. 3. Both are guided through a gap between two heated rolls in order to heat the film of the thermoplastic polymer and force it against the film 3 in order to enclose component 5. An enclosure of component 5 with a filler material 19 is thus produced (the thermoplastic polymer) on film 3, which protects the component 5 from environmental effects, as shown in FIG. 4.

In this step low-temperature sintering, i.e., to form a mechanical and electrical connection 23 between the contact sites 5c and surface 30, can already occur if the rolls are sufficiently warm.

A film of plastic composite material, in the present case glass fibers arranged in an epoxy resin, is now unwound from an additional roll and placed on the arrangement of FIG. 4. Both are guided through a gap between two heated rolls in order to heat the film of the plastic composite material and press it against the enclosure, i.e., the filler material 19 in order to laminate the plastic composite material onto the enclosure.

As shown in FIG. 5, the laminated plastic composite material forms a support 9 for the arrangement of FIG. 4, in the present case rigid support 9. According to one embodiment of the invention low-temperature sintering is conducted in this step to form the mechanical and electrical connection 23 between the contact sites 5c and surface 30, in which case lamination occurs in the temperature range required for this purpose so that sintering is also initiated simultaneously with lamination.

A laser beam directed onto film surface 3u evaporates predetermined sections of film 3. The remaining sections of film 3 form conductor tracks 11 and electrically unbonded cooling section 13, see FIG. 6.

Additional layers 9a and 9b are laminated onto the upper side in FIG. 6, as described above, in which layer 9a is a thermoplastic polymer and layer 9b a plastic composite material, in this case glass fibers arranged in an epoxy resin (see FIG. 7). A cooling channel 15 is formed in these layers by drilling, which is connected to a cooling element 17.

The laminated layers 9, 9a, 9b and 19 with the electrical components 5 situated in them are separated by a laser beam into individual electronic assemblies. A partially broken down perspective view of the assembly 1 produced in this way is depicted in FIG. 8, which is now available for use, for example, in cell phones.

FIG. 9a shows in a perspective view the film 3 with two electronic components 5.1 and 5.2, which are formed here as so-called bare dies.

FIG. 9b shows a top view of film 3 with the components 5.1 and 5.2 fixed on it by the adhesive joint. To form the adhesive joint between component 5.1 and film 3 adhesive 20 is applied along the lateral cutting edge 21 (cf. also FIG. 2), along which component 5.1 was cut out from a wafer, so that the adhesive joint results between the side edge 21 and the surface 30 of film 3.

On the other hand, the adhesive 20 for fixation of component 5.2 on film 3 is applied spot-like in the area of the corners of component 5.2, as also shown in FIG. 9b.

FIG. 9c shows the corresponding sectional views. It is particularly advantageous here in both cases that the possible active surface for electrical contacting of components 5.1 and 5.2 is not reduced or only insignificantly so by formation of the adhesive joint. In particular, despite formation of the adhesive joint, essentially the entire bottom of components 5.1 and 5.2, i.e., the contact side 5b, is available for electrical contacting with film 3 and also the tops of components 5.1 and 5.2.

FIG. 10 schematically depicts a cut-out from a conducting film 3 unwound from a roll according to another embodiment of the invention. This film 3 has adjustment markings 2 at specific, precisely defined sites, for example, in the form of holes passing through film 3. The film 3 is uncoated on both sides. This is a copper film, for example.

Adhesive 20 is applied to film 3 on the upper side 30 in FIG. 10 to FIG. 13 at specified sites that are established in position relative to adjustment markings 2. Application can occur by pressure, injection, dripping or the like. Individual spacer elements 31, for example, glass spheres of a specified size, can be mixed into the adhesive. With the size of the spacers it is possible to adjust different thicknesses of the dielectric. The adhesive also contains nanoparticles to permit subsequent low-temperature sintering, preferably pressureless low-temperature sintering.

In a further process step electronic components are now positioned by means of appropriate devices on the locations at which adhesive 20 was previously applied so that the contact sites 5c of the components come in contact with adhesive 20; this is shown as an example in FIG. 12 for a component 5, which has two contact sites 5c on its contact side 5d.

These electronic components 5 are pressed on so that the adhesive points of the adhesive 20 are squeezed and only cover the contact sites 5c of the electronic components 5 and adhesive it relative to film 3. This state is depicted in FIG. 12. Depending on the type of employed adhesive, additional measures can be taken in order to accelerate curing of the adhesive, for example, additional or exclusive UV curing.

In a subsequent process step, which is not absolutely essential, an enclosure of filler material 19, for example, a polymer mass, can be applied around the electronic components 5, which encloses individual or all electronic components in a separate or common enclosure. Through this process sintering of the nanoparticles contained in adhesive 20 can be initiated in order to produce the joints 23 (cf. FIG. 4), if the process is conducted at a sufficiently high temperature.

The enclosure extends outside the electronic components 5 up to the upper side 30 of film 3 in FIG. 10 to FIG. 13. The adjustment markings 2 can also serve for dimensioning and arrangement of the enclosure of polymer mass relative to the electronic components 5.

Since the enclosure encloses components 5 and extends up to film 3, and optionally also enters into a type of gluing with film 3, a mechanically stable block is formed on this account. The enclosure preferably also extends between the contact sites, as shown in FIGS. 13 and 14.

Subsequent to the process step whose result was shown in FIG. 13 the electronic assembly can now be cut out, i.e., cut out from film 2. The result of the previous process is now reversed so that the free side of film 3 now comes to lie on the top. This is facilitated by the enclosure and the related stabilization of the intermediate product depicted in FIG. 3, especially any required transport between different production stations,

In a further step the film 3 provided with the enclosure is laminated with a circuit board support 9, which comes to lie on the side of film 3 on which the electronic components 5 are also arranged (cf. the arrangement according to FIG. 5). At the latest, sintering and therefore production of the connections 23 is initiated by this.

In a further step the film 3 is now structured on its free side 8 so that conductor tracks are now generated.

This electronic assembly, however, can be further processed and further configured. FIG. 15 shows such a further configuration of electronic assembly in which two assemblies, as shown in FIG. 14, are connected to each other back-to-back with interposition of a metallic layer 12, which is arranged between the two circuit board supports 9. This metallic layer 12 serves, for example, for heat removal of the heat generated in the electronic components 5.

The two joined assemblies here differ from the simple assembly of FIG. 14 also in the fact that an additional metallic conducting layer 14 is applied on the upper metallic layer in FIG. 14, i.e., film 3 with interposition of an insulation layer 2, which produces contacts with conductor tracks of the originally upper conducting layer 2 through openings in the insulation layer 32.

From the nonactive side of the electronic components 5 metallized cooling channels 15 lead through the circuit board support 9 to the middle cooling layer 12 so that the heat can be taken off here.

It is shown as an example in FIG. 15 that the upper of the two layers has electronic components 5 without an enclosure, whereas the lower inverted arrangement contains an enclosure.

An additional embodiment of the method proposed by the invention is depicted in FIG. 16 to FIG. 21. FIG. 16 to FIG. 21 shows states during performance of the method, which roughly correspond to FIG. 10 to FIG. 14.

Whereas in FIG. 10 a film 3 uncoated on both sides is used as starting material, the embodiment depicted in FIG. 16 to FIG. 21 uses a film 33 as starting material, which is applied to a support film 34. The support film 34 can consist of metal, ceramic or also polymer. The conducting film 33, for example, consists of copper and is not coated with nanoparticles.

In a first process step the conducting film 33 is structured so that contact pads 35 are formed on the support film 34, which correspond to the connection sites 5c of the electronic components being attached.

Such an electronic component 5 is shown in FIG. 18. This electronic component 5 is now connected with its connection sites 5c to the contact pads 35. This can occur by applying adhesive 20 to the contact pads 35 or the sides of the connection sites 5c of the electronic component facing them.

The adhesive 20 is an adhesive with nanoparticles distributed in the adhesive. An adhesive layer is therefore formed, which is present between the contact pads 35 and the connection sites 5c of the electronic component 5. In this way the electronic component 5 is connected with its connection sites 5c facing the contact pads 35, that is initially only via adhesive joints.

The electronic component 5 can then be surrounded with an enclosure of a filler material 19, again from a polymer mass, which encloses the entire electronic component 5, including the contact pads 35 and extends to the support film 34. This result is shown in FIG. 19.

The support film 34 can then be separated, which is shown in FIG. 20. Subsequent treatment of the enclosure or lamination of the electronic component contained in it occurs in the usual manner in circuit boards. It is essential that during enclosure with the filler material or the lamination that a temperature range be reached in which low-temperature sintering is initiated for the nanoparticles contained in adhesive 20 in order to produce the connections 23 (cf. FIG. 4).

FIG. 20 shows the attachment of the support layer 34. Instead of detachment of the support layer 34 it is also possible not to detach the support layer 34 but structure it further, for example, by etching, by wear or the like. This is shown in FIG. 21.

FIG. 22 shows a possibility of using a thicker film 36 as initial film, which is then initially structured as in FIG. 17 on its side facing the electronic components in order to form connection pads 35 in so doing. On the left in FIG. 22 the initial state of this thicker film 36 is shown, whereas to the right in FIG. 22 the result of structuring is shown.

The electronic component 5 can then be positioned on the structured film so produced in the same manner, fastened with adhesive 20, as was shown in FIG. 18. Here again structuring can occur after fastening with electronic component 5 and enclosure.

The electronic assemblies, as shown in FIG. 20, FIG. 21 and FIG. 23 can then be assembled in the same manner to multilayer assemblies, as was described in the first embodiment.

The method proposed by the invention makes it possible to achieve significantly increased surface utilization on electronic circuit supports. Additional layers with components positioned in the narrow space can be produced, both active and passive components. The passive and active electronic components can be encapsulated cost effectively so that high reliability is achieved. Risky mixed techniques, namely soldering, gluing and wire bonding can be avoided in manufacture. Through a planar initial structure reproducible HF transitions can be achieved. It is a particular advantage that no separate process step is required to form the electronic connections, since this occurs by low-temperature sintering together with enclosure or lamination and that a particularly stable mechanical connection and an electrical connection with limited contact resistance as well as limited length simultaneously results from this, which is advantageous for high frequency applications.

### LIST OF REFERENCE NUMBERS

1 Electronic assembly
2 Adjustment marking
3 Electrically-conducting film
3a Support film
3o Top of the electrically-conducting film
3u Bottom of the electrically-conducting film
5 Electrical component
5a Protective layer
5b Contact side
5c Electrical contact site
8 Free side
9 Support
9a Support layer
9b Reinforcement layer
11 Conductor tracks
12 Metallic layer
13 Film cooling section
14 Layer
15 Cooling channel
17 Cooling element
19 Filler material
20 Adhesive
21 Edge
22 Top
23 Connection
30 Top side
31 Spacer element
32 Insulation layer
33 Film
34 Support film
35 Contact pads
36 Film
37 Nanoparticles

1. Method for production of electronic assembly (1) with
1.1 Provision of an electrically-conducting film (3), especially support film (3a),
1.2 Provision of at least one electrical component (5) with at least one electrical contact site (5c),
1.3 Application of an adhesive (20) between the electrical component and a surface (30) of the electrically-conducting film,
1.4 Arrangement of the at least one component (5) with at least one electrical contact site (5c) on the surface (30) of the electrically-conducting film (3) and fastening of the at least one component by formation of an adhesive joint between the electrical component and the surface,
1.5 Provision of a support (9), especially from a flexible material,
1.6 Lamination of the film (3) with support (9) so that the at least one electrical component (5) is arranged between film (3) and support (9) and formation of mechanical and electrical connection (23) between the electrical contact site of the at least one electrical component (5) and the electrically-conducting film (3) by low-temperature sintering of nanoparticles, especially from gold, silver, nickel or copper or from an alloy of these metals, in which lamination of the film occurs simultaneously with low-temperature sintering,
1.7 Structuring of the electrically-conducting film (3) to conductor tracks (11) and/or cooling surfaces (13).

2. Method according to claim 1, in which lamination and low-temperature sintering occur at about 130-300° C., especially between 150 and 250° C., especially at 170-190° C., especially 180° C.

3. Method according to claim 1 or 2, in which the adhesive is applied in step 1.3 outside the electrical contact site and the surface of the electrically-conducting film has the nanoparticles.

4. Method according to claim 3, in which the nanoparticles are applied as a full-surface amorphous layer on the surface of the electrically-conducting film.

5. Method according to claim 3 or 4, in which the nanoparticles are applied on the at least one electrical contact site.

6. Method according to claim 3, 4 or 5 in which the adhesive is applied along a lateral edge (21) of the electrical components in order to produce the adhesive joint between the lateral edge and the surface of the electrically-conducting film.

7. Method according to one of the claims 3 to 6, in which the adhesive is applied on one or more of the corners of the electrical component in order to produce the adhesive joint between the corner or corners and the surface of the electrically-conducting film.

8. Method according to claim 1 or 2, in which the adhesive contains nanoparticles and the adhesive is applied to at least one electrical contact site.

9. Method according to claim 8, in which the nanoparticles sintered by low-temperature sintering are formed, which are embedded in a matrix formed by the adhesive.

10. Method according to one of the preceding claims, in which low-temperature sintering is conduced as pressureless low-temperature sintering.

11. Method according to one of the preceding claims, in which the nanoparticles have a particle size from 20 to 10,000 nm, especially 300-3000 nm.

12. Method according to one of the preceding claims, in which the nanoparticles are silver with a particle size between 20 and 300 nm.

13. Method according to one of the preceding claims, in which the surface of the film is provided with a uniform coating of about 200-300 nm silver or silver-containing alloy.

14. Method according to one of the preceding claims, in which supply of the film (3) in step 1.1 occurs by supplying individual, cut-out film sections or by supplying an endless film from a roll.

15. Method according to one of the preceding claims, in which supply of the at least one electrical component (5) in step 1.2 occurs by supplying it on an endless film or supply in a magazine.

16. Method according to one of the preceding claims, in which arrangement of the at least one electrical component (5) in step 1.4 occurs in automated fashion by insertion robot or by hand, especially by means of templates or position markers in or on the film.

17. Method according to one of the preceding claims, in which supply of the support (9) in step 1.5 occurs by supplying individual, cut-out support sections or by supplying an endless support from a roll.

18. Method according to one of the preceding claims, in which lamination of the film (3) with support (9) in step 1.6 occurs by exerting a pressure on film (3) in the direction of support (9), especially with simultaneous effect of heat.

19. Method according to one of the preceding claims, in which structuring of the electrically-conducting film (3) to conductor tracks (11) and optionally cooling surfaces (13) in step 1.7 occurs by direct structuring, for example, with a laser or by generating a positive or negative mask and subsequent etching.

20. Method according to one of the preceding claims, in which formation of the connection (7) in step 1.6 occurs in a vacuum or an atmosphere of protective gas.

21. Method according to one of the preceding claims, characterized by winding of the film (3) with support (9) and the at least one electrical component (5) after step 1.7 on a roll.

22. Method according to one of the preceding claims, characterized by enclosure of the at least one electrical component after step 1.4 with a filler material (19) from a polymer, especially a thermoplastic, thermosetting plastic or elastomer, especially a liquid crystal polymer (LCP).

23. Method according to one of the preceding claims, characterized by filling of the filler material (19) in a predefined form, especially by casting, foaming, extrusion or lamination.

24. Method according to one of the preceding claims, characterized by generation of at least an additional support layer (9a) on the conductor tracks (11) generated in step 1.7, which preferably is insulating and/or dielectric and/or contains additional conductor tracks.

25. Method according to one of the preceding claims, characterized by the fact that after step 1.7 application of at least one reinforcement layer (9b), preferably several reinforcement layers (9b), occurs on the support (9) of the support layer (9a) or that the support (9) is optionally removed after step 1.6.

26. Method according to one of the preceding claims, characterized by cutting out of support (9) in a predetermined shape to form electronic assembly (1), especially before and/or after lamination with the support (9) in step 1.6.

27. Method for production of electronic assembly (1) with
1.1 Supply of an electrically-conducting film (3), especially support film (3a),
1.2 Supply of at least one electrical component (5) with at least one electrical contact site (5c),
1.3 Application of an adhesive (20) between the electrical component and a surface (30) of the electrically-conducting film,
1.4 Arrangement of the at least one component (5) with the at least one electrical contact site (5c) on surface (30) of the electrically-conducting film (3) and fastening of the at least one component by formation of an adhesive joint between the electrical component and the surface by the applied adhesive, and closure of the at least one electrical component with a filler material (19) from a polymer, especially a thermoplastic, thermosetting plastic or an elastomer, especially a liquid crystal polymer (LCP) and formation of a mechanical and electrical connection (23) between the electrical contact site of the at least one electrical component (5) and the electrically-conducting film (3) by low-temperature sintering, in which enclosure occurs simultaneously with low-temperature sintering,
1.5 Supply of the support (9), especially from a flexible material,
1.6 Lamination of the film (3) with support (9) so that the at least one electrical component (5) is arranged between film (3) and support (9),
1.7 Structuring of the electrically-conducting film (3) to conductor tracks (11) and/or cooling surfaces (13).

28. Method according to claim 27, in which enclosure and low-temperature sintering occur by positioning of the film of the thermoplastic polymer on the fixed components and the electrically-conducting film with the components fastened to it and the applied film of the thermoplastic polymer is then passed through a gap between two heated rolls.

29. Method according to claim 27 or 28, in which lamination and low-temperature sintering occur at about 130-300° C., especially between 150 and 250° C., especially at 170-190° C., especially at 180° C.

30. Method according to claim 27, 28 or 29, in which the adhesive in step 1.3 is applied outside the electrical contact site and the surface of the electrically-conducting film has the nanoparticles.

31. Method according to claim 30, in which the nanoparticles are applied as a full-surface amorphous layer on the surface of the electrically-conducting film.

32. Method according to claim 30 or 31, in which the nanoparticles are applied to the at least one electrical contact site.

33. Method according to claim 30, 31 or 32 in which the adhesive is applied along a side edge (21) of the electrical component in order to produce the adhesive joint between the side edge and the surface of the electrically-conducting film.

34. Method according to one of the claim 30, 31 or 32, in which the adhesive is applied on one or more of the corners of the electrical component in order to produce the adhesive joint between the corner or corners and the surface of the electrically-conducting film.

35. Method according to claim 27, 28 or 29, in which the adhesive contains nanoparticles and the adhesive is applied to at least one electrical contact site.

36. Method according to claim 35, in which the nanoparticles sintered by low-temperature sintering are formed, which are embedded in a matrix formed by the adhesive.

37. Method according to one of the preceding claims, in which low-temperature sintering is conduced as pressureless low-temperature sintering.

38. Method according to one of the preceding claims, in which the nanoparticles have a particle size from 20 to 10,000 nm, especially 30-3000 nm.

39. Method according to one of the preceding claims, in which the nanoparticles are silver with a layer between 200 and 300 nm.

40. Electronic assembly produced according to the method according to one of the claims 1 to 39.

41. Electronic assembly, especially implant for implantation of the human or animal body with
an electrically-conducting film (3), especially a support film (3a),
at least one electrical component (5) of at least one electrical contact site (5c),
an adhesive joint between the electrical component and a surface of the electrically-conducting film,
a support (9), especially from a flexible material,
a film (3) laminated with the support, in which the at least one electrical component (5) is arranged between film (3) and support (9) and a mechanical and electrical sinter connection (23) between the electronic contact site of the at least one electronic component (5) and the electrically-conducting film (3) in which the sinter connection is formed by sintered nanoparticles, especially from gold, silver, nickel or copper or from an alloy of these metals and in which the electrically-conducting film (3) is structured to conductor tracks (11) and/or cooling surfaces (13).

42. Electronic assembly according to claim 41, in which the sinter connection in the adhesive joint is formed by embedding sintered nanoparticles in a matrix formed by the adhesive joint, especially a resin matrix.

43. Electronic assembly according to claim 41, in which surface (30) of the electrically-conducting film is coated with the nanoparticles and the adhesive joint runs outside the at least one electrical contact site.
